SEQUENCE LISTING (2 sequences)

SEQ ID NO: 1 - protein, 58 aa, Bos taurus
RPDFCLEPPYTGPCKARMIRYFYNAKAGLCQPFVYGGCRAKRNNFKSSEDCMRTCGGA

SEQ ID NO: 2 - protein, 9 aa, Homo sapiens
CYIQNCPLG

(19) United States
(12) Patent Application Publication
(10) Pub. No.: US 2025/0152706 A1
(43) Pub. Date: May 15, 2025

(54) COMPOSITIONS FOR ORAL DELIVERY OF BIOTHERAPEUTICS

(71) Applicant: INTRACT PHARMA LIMITED, London (GB)

(72) Inventor: Vipul YADAV, London (GB)

(73) Assignee: INTRACT PHARMA LIMITED, London (GB)

(21) Appl. No.: 18/832,134

(22) PCT Filed: Feb. 9, 2023
(86) PCT No.: PCT/EP2023/053219

§ 371 Date: Jul. 23, 2024

(30) Foreign Application Priority Data
Feb. 10, 2022 (GB) 2201708.1

(51) Int. Cl. A61K 39/395; A61K 31/197; A61K 38/36

(52) U.S. Cl. CPC A61K 39/3955; A61K 31/197; A61K 38/36

(57) ABSTRACT

The present invention relates to methods of stabilising proteins and peptides in the lower gastrointestinal tract. Pharmaceutical compositions which comprise as active ingredient a protein or a peptide; 5-aminolevulinic acid (5-ALA) or a pharmaceutically acceptable salt thereof; and optionally a pH-modulating component and/or an enzyme inhibitor are described

### FIELD OF THE INVENTION

This invention relates to novel compositions and their use.

### BACKGROUND

Biopharmaceuticals, and particularly proteins including antibodies and peptides, are an increasingly important class of pharmaceuticals, and many therapeutic uses have been proposed and implemented. Whereas the most convenient and patient-preferred mode of drug administration is oral, because of the instability of this type of molecule in the gastrointestinal (GI) tract and the inefficiency in traversing GI membranes, almost all biopharmaceuticals are currently delivered by injection.

There is an unmet need for a method of sufficiently stabilising proteins which are used as biotherapeutics, in the presence of luminal fluid found in the lower gastrointestinal tract, such as the small intestine, and/or the colon for them to be delivered for either local or systemic delivery via the ileum and/or the colon by oral administration.

Further, to optimise systemic delivery following oral administration it is necessary to provide a means to facilitate uptake through the membranes of the GI tract and into systemic circulation. Many such “penetration enhancing” compounds and methodologies have been described.

Previously it has been demonstrated that proteins can be protected from breakdown in the GI tract by formulation in the presence of certain protective excipient molecules. In particular, it has been demonstrated that antibodies can be protected from digestion in the colonic lumen by co-formulation with enzyme-inhibiting molecules such as aprotinin in combination with certain dipeptide molecules (WO2020/229693). It has further been demonstrated that peptides, in particular cyclic peptides and peptides containing non naturally-occurring amino acids (D-amino acids) can be stabilised in GI fluids, particularly colonic fluids, by formulating in the presence of certain amino acids (WO2022/101322).

The present inventors have found that formulation of a protein or peptide with the non-proteinogenic amino acid 5-aminolevulinic acid (5-ALA) results in increased stability of the protein or peptide in body fluids found in the lower gastrointestinal tract, such as the small intestine and/or the colon. Addition of a pH-modulating component to increase the pH of the mixture, and/or an enzyme inhibitor can serve to further enhance stability of the protein or peptide.

The inventors have additionally shown that combining 5-ALA in a formulation with a peptide or protein results in increased permeation of the protein or peptide into and through the membranes of the GI tract, allowing for the possibility of greater uptake of the protein or peptide into GI tissue or through GI tissue and into systemic circulation.

### SUMMARY OF THE INVENTION

Accordingly, the present invention provides a pharmaceutical composition which comprises, consists, or consists essentially of a protein or a peptide as active ingredient; 5-aminolevulinic acid (5-ALA) or a pharmaceutically acceptable salt thereof; and optionally a pH-modulating component and/or an enzyme inhibitor.

Preferably, if the active ingredient is a protein, the composition comprises consists, or consists essentially of the protein active ingredient, 5-ALA or a pharmaceutically acceptable salt thereof; and a pH-modulating component and/or an enzyme inhibitor. Preferably the protein active ingredient is an antibody or fragment thereof.

Preferably, if the active ingredient is a peptide, the composition comprises, consists, or consists essentially of the peptide active ingredient, 5-ALA or a pharmaceutically acceptable salt thereof; and an enzyme inhibitor. Preferably the peptide active ingredient is a linear or a cyclic peptide.

The protein may be an antibody or fragment thereof. The peptide may be a linear or a cyclic peptide.

The composition of the invention may be in liquid or in solid or in semi-solid form, preferably in a form suitable for rectal or, especially, oral administration. The compositions may be in a solid form suitable for oral administration, said composition having an enteric coating. Most preferably it is in a solid or semi-solid form suitable for oral administration, and adapted for selective release of the protein in the lower gastrointestinal tract, in particular the ileum and/or the colon.

The composition may comprise a solid dosage form with a core and a coating for the core, the core comprising as an active ingredient a protein or peptide, 5-ALA and optionally a pH-modulating component and/or an enzyme inhibitor; and the coating comprising a mixture of a digestible polysaccharide and a film-forming material which has a solubility threshold at pH 6.0 or above, i.e. it dissolves at a pH of 6.0 or above.

The composition may be an orally administrable pharmaceutical composition comprising (a) as active ingredient a protein or a peptide and (b) 5-aminolevulinic acid (5-ALA) or a pharmaceutically acceptable salt thereof, and optionally (c) a pH-modulating component and/or (d) an enzyme inhibitor.

The invention also provides a solid dosage form for oral administration comprising a core comprising (a) as active ingredient a protein or a peptide and (b) 5-aminolevulinic acid (5-ALA) or a pharmaceutically acceptable salt thereof, and optionally (c) a pH-modulating component and/or (d) an enzyme inhibitor.

The invention also provides a rectally administrable pharmaceutical composition comprising (a) as active ingredient a protein or a peptide and (b) 5-aminolevulinic acid (5-ALA) or a pharmaceutically acceptable salt thereof, and optionally (c) a pH-modulating component and/or (d) an enzyme inhibitor.

The invention also provides an enema formulation comprising (a) as active ingredient a protein or a peptide and (b) 5-aminolevulinic acid (5-ALA) or a pharmaceutically acceptable salt thereof, and optionally (c) a pH-modulating component and/or (d) an enzyme inhibitor.

The present invention further provides a pharmaceutical composition according to the invention for use in therapy. Further, there is also provided a pharmaceutical composition according to the invention for use in the treatment or prevention of disease or conditions selected from an inflammatory bowel disease (IBD); irritable bowel syndrome; constipation; diarrhoea; infection; or cancer. Preferably the pharmaceutical composition is a pharmaceutical composition of the invention. Preferably the active ingredient is an antibody.

The invention further provides a method of treating or preventing a disease or condition in a subject which comprises administering to the subject a pharmaceutical composition according to the invention. Preferably, the disease or condition is selected from inflammatory bowel disease; irritable bowel syndrome; constipation; diarrhoea; infection; autoimmune disease or cancer. Preferably the pharmaceutical composition is a pharmaceutical composition of the invention. Preferably the active ingredient is an antibody.

The present invention further provides a method of stabilising a protein or peptide in the presence of intestinal fluid, which comprises delivering an active ingredient selected from a protein or a peptide with 5-ALA and optionally a pH modulating agent and/or an enzyme inhibitor.

The invention also provides the use of 5-ALA, optionally with a pH modulating component and/or an enzyme inhibitor to stabilise a protein or peptide administered as a pharmaceutically active ingredient and delivered to the lower gastro-intestinal tract. Preferably the pharmaceutical composition is a pharmaceutical composition of the invention.

Following release, penetration of the drug into GI tissue at or close to the site of release in the GI lumen can occur, allowing for local treatment of diseases of the GI tract, and, through penetration of the drug into the bloodstream, treatment of a large range of diseases.

The invention also provides the use of 5-ALA, optionally with a pH modulating component and/or an enzyme inhibitor to improve penetration of a protein or peptide into the tissue lining the GI tract, when the protein or peptide is administered as a pharmaceutically active ingredient in a pharmaceutical composition and delivered to the lower gastro-intestinal tract. Preferably the pharmaceutical composition is a pharmaceutical composition of the invention.

### DETAILED DESCRIPTION OF THE INVENTION

As used herein “lower gastrointestinal tract” refers to gastrointestinal tract after the stomach. This includes the small intestine and large intestine. The small intestine is made up of the duodenum, jejunum and ileum, while the large intestine is also known as the colon. Preferably, the stabilising agents (for example 5-ALA, the pH-modulating component and/or enzyme inhibitor) reduce degradation of the protein or peptide in the ileum and/or colon.

### The Protein or Peptide

The protein or peptide may be any protein or peptide having a beneficial therapeutic effect towards human or animals, whose therapeutic effect is advantageously realised by administration via the lower gastrointestinal tract, preferably to the ileum and/or the colon, especially the colon.

Classes of proteins for use in the present invention include for example antibodies as described below, enzymes, cytokines, chemokines, receptors, blood factors, peptide hormones, toxins, transcription proteins, non-immunoglobulin binding protein scaffolds, and multimeric proteins.

### Antibodies

As used herein, “antibody” means an immunoglobulin molecule that recognizes and specifically binds to a target antigen, such as a cytokine, protein, polypeptide, peptide, carbohydrate, polynucleotide, lipid, bacteria or virus, or combination thereof through at least one antigen recognition site within the variable region of the immunoglobulin molecule. The term “antibody” encompasses polyclonal antibodies, monoclonal antibodies, multispecific antibodies such as bispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. Preferably the antibody is a monoclonal antibody. An antibody can include any of the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Preferably, the antibody is an IgG antibody, more preferably an IgG1 or IgG4. The term “antibody” is also intended to include conjugates of the antibody, for example conjugates with polyethylene glycol, PEG.

Further, except where the context requires otherwise, the term “antibody” should be understood to encompass complete antibodies and antibody fragments comprising an antigen-binding region of the complete antibody. Antibody fragments may for example be single domain antibodies (e.g. V_{H}H domain antibodies) monovalent or divalent Fab, Fab′, F(ab′)2, scFv, Fc, bispecific antibodies, diabodies, minibodies or multispecific antibodies formed from antibody fragments, for example minibodies composed of different permutations of scFv fragments or diabodies, and optionally Fc fragments or CH domains, such as scFv-Fc, scFv-Fc-scFv, Fab-scFv, (Fab′ScFv)2, scDiabodies, scDiabody-Fc, scDiabody-CH3, scFv-CH3, and scFv-CH2-CH3 fusion proteins. An antibody fragment can be produced by enzymatic cleavage of a complete antibody, or by synthetic means such as recombinant DNA techniques, phage display or yeast display technologies or using transgenic mice, or liquid or solid phase peptide synthesis.

Where an antibody is used in a composition according to the invention, it may be any one whose therapeutic effect is advantageously realised by administration via the colon. Specific antibodies of particular interest in the context of the present invention include existing commercial IBD therapeutic antibodies such as adalimumab, infliximab, cetrolizumab pegol, golimumab, natalizumab, vedolizumab, tildrakizumab, ustekinumab, and additional antibodies in development for IBD treatment which target pathways and molecules (agonists or antagonists) implicated in pathogenesis of IBD, such as, for example CD40. Targeting to the colon additionally affords the possibility to improve treatment of colorectal cancer by targeting and localization of anti-cancer therapeutic antibodies, or of different possible formats as mentioned above, to the tumour.

### Peptides

Peptides are chains of amino acid monomers joined together by amide bonds. Peptides can be made up of two or more amino acid monomers. Preferably the peptides contain less than 50 amino acid monomers, preferably 2-50 amino acid monomers, more preferably 2-10 amino acid monomers, 5-20 amino acid monomers, 15-40 amino acid monomers or 18-30 amino acid monomers. Preferably the peptides have a molecular weight up to 5 kDa, more preferably between 1-2.5 kDa. Suitable peptides include linear, branched and cyclic peptides with disulphide bridges. Cyclic peptides may include 1-5 disulfide bridges, preferably 1-3 disulfide bridges. Preferably the peptide is a cyclic peptide.

The peptides are generally made from naturally occurring L form amino acids. Variant peptides may contain one or more D-form and/or non-natural amino acids, such as those with modified side groups. Preferably the variant peptide has one, two, three, four or five D form amino acids. Preferably in a variant peptide, an L form amino acid is replaced by the equivalent D form amino acid, or the equivalent amino acid with a modified side group. In addition, variant forms of the peptide may be linear or cyclic versions of normally cyclic or linear peptides respectively.

One preferred peptide is oxytocin and variants thereof. Oxytocin has the amino acid sequence Cys-Tyr-Ile-Gln-Asn-Cys-Pro-Leu-Gly (SEQ. ID NO: 2), and usually contains a sulfide bridge between the cysteine residues.

### Stabilising Agents

### 5-Aminolevulinic Acid

As used herein, “5-ALA” refers to the naturally occurring amino acid 5-aminolevulinic acid, precursors, derivatives or a pharmaceutically acceptable salts thereof. The compound, 5-aminolevulinic acid, described below, has previously been utilized as a precursor of a photosensitizer PpIX for performing photodynamic diagnosis and therapy to confirm and kill tumor cells. 5-ALA is converted into PpIX and heme in the mitochondria with the insertion of iron catalysed by ferrochelatase. HO-1 further catalyses the conversion of heme into carbon monoxide (CO), biliverdin and bilirubin. 5-ALA (CAS No. 106-60-5) has the general formula as below:

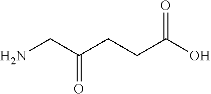

A “pharmaceutically acceptable salt” is one which is safe for use in mammals and retain the desired biological activity. Suitable salts include but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Preferably, the salt is hydrochloride.

“5-ALA” as used herein also encompasses precursors of 5-aminolevulinic acid. A “precursor” is a compound which is metabolised to 5-aminolevulinic acid following administration in the intestinal tract, e.g. through the action of bacteria found in the gastrointestinal tract, in particular the lower gastrointestinal tract.

“5-ALA” as used herein also encompasses derivatives of 5-aminolevulinic acid and their pharmaceutically acceptable salts. Derivatives include esters, where the —OH group of 5-aminolevulinic acid is replaced with —O—R wherein R is an alkyl group with up to 8 carbons atoms. Preferably the alkyl group has 1, 2, 3, 4, 5, 6, 7 or 8 carbon atoms. The alkyl group is preferably unbranched. Preferably the alkyl group is methyl or hexyl. Suitable derivative esters include methyl aminolevulinic acid and hexyl aminolevulinic acid, and preferred salts include methyl aminolevulinate hydrochloride and hexyl aminolevulinate hydrochloride.

5-ALA optionally in combination with a pH-modulating agent, and/or an enzyme inhibitor acts as a stabilising agent which help to maintain the intact protein or peptide in the lower gastrointestinal tract. Methods for assessing the stability of the protein or peptide in gastrointestinal fluid are described in the Examples.

5-ALA optionally in combination with a pH-modulating agent, and/or an enzyme inhibitor cause at least 5% of the peptide or protein present to remain intact after 1 hour in the gastrointestinal fluid. Preferably at least 10%, 20%, 30%, 40%, 50%, 60%, 70%, 75%, 80%, 90%, 95% or more of the peptide or protein remains intact.

pH-Modulating Component

The pH modulating component is one which alters the pH of gastric fluid as compared to the gastric fluid containing the administered 5-ALA. The pH modulating component neutralises the effect of the 5-ALA so that the pH of the gastrointestinal fluid does not alter when composition is administered. Preferably the pH modulating component increases the pH, so that the gastric fluid has a pH around 6.0 after addition of the composition. The pH modulating component is preferably a base. Suitable pH modulating components include arginine, sodium hydroxide, ammonia, ammonium carbonate, diethanolamine, monoethanolamine, potassium hydroxide, sodium bicarbonate, sodium borate, sodium carbonate, and trolamine. Preferably the pH modulating component is arginine.

### Enzyme Inhibitor

Preferably the enzyme inhibitor is a protease inhibitor, such as for example, aprotinin, Ovomucoid type II-O (containing ovoinhibitor), Bowman-Birk inhibitor (BBI), or Kunitz trypsin inhibitor for protection of proteins.

Preferably the enzyme inhibitor is aprotinin or an analogue thereof.

### Aprotinin

The pharmaceutical composition of the invention preferably comprises aprotinin, or a fragment, or an analogue of aprotinin. It will of course be understood that aprotinin, fragments or analogues of aprotinin are not present in the composition of the invention as an active ingredient.

Preferably, the composition does not comprise a combination of aprotinin or an analogue of aprotinin together with (a) carnitine, an acyl carnitine, or a salt of carnitine or an acyl carnitine; (b) a salt or an ester of a bile acid; or (c) an alkylsaccharide.

Aprotinin is a protease inhibitor, which is known to inhibit trypsin and other similar proteases. It is often also referred to as Bovine Pancreatic Trypsin Inhibitor (BPTI). Aprotinin is a 58 amino acid protein that is formed after processing of a 100 amino acid polypeptide that comprises a signal peptide, propeptide domains, and a Kunitz domain. Aprotinin in its mature 58 amino acid form is available commercially and is also sold under the trade name Trasylol®, which is indicated for prophylactic use to reduce blood loss during surgery.

The aprotinin for use in the invention may comprise the amino acid sequence

(SEQ. ID NO: 1)
RPDFCLEPPYTGPCKARMIRYFYNAKAGLCQPFVYGGCRAKR
NNFKSSEDCMRTCGGA.

The aprotinin, fragment or aprotinin analogue for use in the invention may be prepared recombinantly (for example, in E. coli, mammalian cells or insect cells), synthetically (for example, using standard organic chemistry techniques, such as solution or solid phase peptide synthesis), or it may be a native protein from an animal source, such as a bovine source.

Further, except where the context requires otherwise, the term “aprotinin” should be understood to encompass aprotinin, analogues of aprotinin and fragments of aprotinin. Suitable analogues and fragments of aprotinin for use in the present invention are those which when combined with 5-ALA enhance the stability of a protein or peptide in the presence of the body fluids found in the lower gastrointestinal tract, for example the ileum and/or the colon. Methods for confirming the ability of aprotinin, a fragment of aprotinin or an analogue of aprotinin to enhance the stability of a protein or peptide in the presence of the body fluids found in the lower gastrointestinal tract, such as the ileum and/or the colon are described in the Examples below. “Enhanced” stability as used herein means that at least 15% of the peptide or protein present remains intact after 1 hour, preferably 2 hours, in the gastrointestinal fluid. Preferably at least 20%, 25%, 30%, 40%, 50%, 60%, 70%, 75%, 80%, 90% 95% or more of the peptide or protein remains intact.

For example, aprotinin fragments for use in the present invention may comprise a fragment of the sequence defined by SEQ ID NO 1. For example, the aprotinin fragment may comprise at least 30, 40, 50, 51, 52, 53, 54, 55, 56 or 57 contiguous amino acids of the sequence defined by SEQ ID NO 1.

Analogues of aprotinin for use in the present invention may include proteins comprising a sequence similar to the amino acid sequence defined in SEQ. ID NO: 1, and which when combined with 5-ALA enhance the stability of a peptide or protein in the presence of the body fluids found in the lower gastrointestinal tract, such as the ileum and/or the colon. For example, the aprotinin analogue may have at least 70%, 80%, 90% or at least 95% similarity to the sequence defined in SEQ. ID NO: 1 or a fragment of the sequence defined by SEQ. ID NO: 1. Alternatively, the aprotinin analogue may have at least 70%, 80%, 90% or at least 95% identity to the sequence defined in SEQ. ID NO: 1 or a fragment of the sequence defined by SEQ. ID NO: 1. For example, the aprotinin analogue may comprise an amino acid sequence that is identical to 50, 51, 52, 53, 54, 55, 56 or 57 amino acids of the sequence defined by SEQ. ID NO: 1. The similar or identical amino acids may be contiguous or non-contiguous.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of SEQ. ID No:1.

The analogue of aprotinin may contain one or more amino acid substitutions, insertions and/or deletions.

Amino acid substitution means that an amino acid residue is substituted for a replacement amino acid residue at the same position. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, lie, Val, Cys; Group 5 Phe Thy Trp.

Inserted amino acid residues may be inserted at any position and may be inserted such that some or all of the inserted amino acid residues are immediately adjacent one another or may be inserted such that none of the inserted amino acid residues is immediately adjacent another inserted amino acid residue. For example, the aprotinin analogue may comprise 1, 2, 3, 4, 5, 6, 7, 8, 9 or 10 additional amino acids at the N- and/or C-terminus of the amino acid sequence defined in SEQ. ID NO: 1.

One, two or three amino acids may be deleted from the sequence of SEQ. ID NO: 1. Each deletion can take place at any position of SEQ. ID NO: 1.

Inserted amino acids and replacement amino acids may be naturally occurring amino acids or may be non-naturally occurring amino acids and, for example, may contain a non-natural side chain, and/or be linked together via non-native peptide bonds. Such altered peptide ligands are known in the art. If more than one amino acid residue is substituted and/or inserted, the replacement/inserted amino acid residues may be the same as each other or different from one another. Each replacement amino acid may have a different side chain to the amino acid being replaced.

Analogues of aprotinin may comprises one or more modified bases, wherein the amino acid residues may be chemically modified. Examples of chemical modifications include those corresponding to post translational modifications for example phosphorylation, acetylation and deamidation. Chemical modifications may not correspond to those that may be present in vivo. For example, the N or C terminal ends of the aprotinin peptide may be modified improve the stability, bioavailability and or affinity of the peptides. Further examples of non-natural modifications include incorporation of non-encoded α-amino acids, photoreactive cross-linking amino acids, N-methylated amino acids, and β-amino acids, backbone reduction, retroinversion by using d-amino acids, N-terminal methylation and C-terminal amidation and pegylation.

### Pharmaceutical Formulations

The pharmaceutical compositions according to the invention are preferably in solid or semi-solid form, and preferably they are suitable for oral or rectal administration.

The composition may also be in the form of a lotion, cream, foam, emulsion or gel. Such formulations may be prepared by a number of known methods established in the art.

For example, the protein or peptide and the required excipient may be admixed together, optionally together with other excipients required in the dosage form.

Pharmaceutical compositions in the present invention that are suitable for oral administration may be presented either in the form of tablets, capsules, mini-tablets, pellets, powders, granules, microparticles, nanoparticles or hydrogels.

Compositions of the present invention suitable for oral administration may be presented as discrete units such as capsules, tablets, mini-tablets, or pellets, or as powders, granules or crystals. In a solid composition, the minimum diameter of each particle is typically at least 10⁻⁴ m, usually at least 5×10⁻⁴ m and, preferably at least 10⁻³ m. The maximum diameter is usually no more than 30 mm, typically no more than 20 mm and, preferably, no more than 10 mm. In preferred embodiments, the particle has a diameter from about 0.2 mm to about 15 mm, preferably from about 1 mm to about 4 mm (e.g. for pellets or mini-tablets) or from about 6 mm to about 12 mm (e.g. for certain tablets or capsules). The term “diameter” refers to the largest linear dimension through the particle.

As well as the required 5-ALA or a pharmaceutically acceptable salt thereof, and optional a pH-modulating component and/or an enzyme inhibitor, compositions according to the invention may of course contain any further conventional excipients as required such as binders, extenders, disintegrants, diluents and lubricants. Excipients used in solid forms include for example, microcrystalline cellulose, dicalcium phosphate, starch, magnesium stearate, calcium sulfate, sorbitol, glucose and/or lactose and/or other excipients, binders, extenders, disintegrants, diluents and lubricants known in the art. Suitable binders include starch, gelatine, natural sugars such as glucose or beta-lactose, corn sweeteners, natural and synthetic gums such as acacia, tragacanth or sodium alginate, carboxymethylcellulose, polyethylene glycol, waxes and the like. Disintegrators include without limitation starch, methylcellulose, agar, bentonite, xanthan gum and the like. Fast dissolving diluents include mannitol, lactose, sucrose and/or cyclodextrins. Lubricants, glidants, flavours, colouring agents and stabilizers may also be added for ease of fabrication and use. Lubricants include sodium oleate, sodium stearate, magnesium stearate, sodium benzoate, sodium acetate, sodium chloride.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow, delayed or controlled release of the antibody. Preferred examples of coatings are given below.

Capsules may have solid, semi-solid or non-solid contents. Exemplary contents for capsules may include suspensions which can contain, for example, microcrystalline cellulose for imparting bulk, alginic acid or sodium alginate as a suspending agent, and methylcellulose as a viscosity enhancer, as well as any of the solid or semi-solid forms above.

Formulations for rectal administration may be presented as a suppository with the usual carriers such as cocoa butter, synthetic glyceride esters or polyethylene glycol. Such carriers are typically solid at ordinary room temperatures (up to 25° C.), but liquefy and/or dissolve in the rectal cavity to release the drug.

Compositions may also take the form of an enema formulation such as a liquid or foam enema which is rectally administered to the lower colon. The enema formulations typically comprise the peptide or protein, together with the 5-ALA, optionally in combination with a pH-modulating agent, and/or an enzyme inhibitor, dissolved or dispersed in a suitable flowable carrier vehicle, such as deionised and/or distilled water. The formulation can be thickened with one or more thickeners. They may also contain a buffer, and can also comprise an effective amount of a lubricant such as a natural or synthetic fat or oil, e.g. a tris-fatty acid glycerate or lecithin. Non-toxic non-ionic surfactants can also be included as wetting agents and dispersants. A buffer is preferably added to the liquid or foam enema to stabilise the pH. The pH of the enema formulation is preferably 3.5 to 7.5, especially 6.5 to 7.5.

Unit doses of enema formulations can be administered from pre-filled bags or syringes. In the case of a pressurised enema formulation the carrier vehicle may also comprise an effective amount of a foaming agent such as n-butane, propane or i-butane, or the foaming agent/propellant could be held separately from the composition such as in a bag-in-bag or bag-in-can system as described in WO-A-9603115 (incorporated herein by reference). Enema foams may also comprise expanding agents and foam-stabilisers.

The volume of a liquid enema is typically 50-200 cm³, preferably about 100 cm³. The volume of a foam enema is typically 20 to 40 cm³. A suitable dosage of the 5-ALA or pharmaceutical salt thereof in the enema as administered is 1 mg/ml to 20 mg/ml, preferably 2 mg/ml to 10 mg/ml.

Preferred unit dosage formulations are those containing an effective dose, or an appropriate fraction thereof, of the active ingredient. Release from certain formulations may also be sustained, if the composition contains suitable controlled-release excipients. However, in preferred formulations, release is pulsatile.

The compositions according to the invention will typically comprise a therapeutically effective amount of the protein or peptide which may be from 0.01 wt % to 99 wt %, based on the total weight of the composition. The actual dosage would be determined by the skilled person using common general knowledge. However, by way of example, “low” dose formulations typically comprise no more than 20 wt % of the protein or peptide, and preferably comprise from 1 wt % to 10 wt %, e.g. 5 wt %, of the protein or peptide. “High” dose formulations typically comprise at least 40 wt % of the protein or peptide, and preferably from 45 wt % to about 85 wt %, e.g. 50 wt % or 80 wt %.

The protein or peptide active agent is typically present in an amount resulting in a concentration of 0.1-25 mg/ml in the lower gastrointestinal tract. Each dosage form may contain 50-5000 mg protein or peptide, typically 100-1000 mg; 250-750 mg or 300-500 mg.

The compositions according to the invention will typically comprise an effective amount of stabilising agents (i.e. total amount of 5-ALA or pharmaceutically acceptable salt thereof, and if present pH-modulating agent and/or enzyme inhibitor). Typically, formulations comprise 0.01 wt % to 99 wt % of the stabilising agents, based on the total weight of the composition The compositions may preferably comprise no more than 20 wt % of the stabilising agents, and preferably comprise from 1 wt % to 10 wt %, e.g. 5 wt %, of the stabilising agents. Alternatively, the compositions may comprise at least 40 wt % of the stabilising agents, and preferably from 45 wt % to about 85 wt %, e.g. 50 wt % or 80 wt %.

5-ALA is typically present in an amount resulting in a concentration of 0.1-25 mg/ml in the lower gastrointestinal tract. Each dosage form may contain 50-5000 mg 5-ALA, typically 100-1000 mg; 250-750 mg or 300-500 mg.

If included in the formulation, the enzyme inhibitor, such as aprotinin, is typically present in an amount resulting in a concentration of 0.1-25 mg/ml in the lower gastrointestinal tract. Each dosage form may contain 50-5000 mg aprotinin, typically 100-1000 mg; 250-750 mg or 300-500 mg.

If included in the formulation, the pH-modulating agent, such as arginine, is typically present in an amount resulting in a pH in the range of 5.5-7.5 in the lower gastrointestinal tract. Typically, the pH-modulating agent is present at a concentration of 0.1-25 mg/ml in the lower gastrointestinal tract. Each dosage form may contain 50-5000 mg aprotinin, typically 100-1000 mg; 250-750 mg or 300-500 mg.

Whilst the protein or peptide may be used as the sole active ingredient in a composition according to the invention, it is also possible for the protein or peptide to be used in combination with one or more further therapeutic agents such as an additional protein or peptide, or a non-biologic drug. Thus, the invention also provides a composition according to the invention containing a further therapeutic agent in addition to the protein or peptide. If desired, the composition according to the invention may be administered together with a further composition, by simultaneous, sequential or separate administration.

Except where the context requires otherwise, throughout this Specification and claims, any reference to a pharmaceutical composition in solid or semi-solid form should be understood to include individual solid or semi-solid particles or unit forms which are solid or semi-solid throughout, as well as those having a solid or semi-solid exterior and a non-solid, for example liquid or gel, interior. For example, a capsule may have liquid or gel contents.

### Delivery to the Lower Gastrointestinal Tract

The composition according to the invention is adapted for delayed or selective release of the protein or peptide in the lower gastrointestinal tract, in particular the ileum and/or, especially, the colon, suitably following rectal or, especially, oral administration. This may be accomplished by the use of particular coatings. The compositions of the invention may be delayed release oral (DRO) compositions. The DRO compositions pass through the stomach substantially unaltered and deliver the active ingredient to the lower gastrointestinal tract, typically the ileum and/or colon (i.e. the site of the diseased mucosa).

The compositions according to the invention may have an enteric coating. Enteric coatings protect the active ingredients in a composition from attack and degradation in the stomach, but dissolve and release to contents of the dosage form within the intestines, usually due to the change in pH. Suitable enteric coatings are well known in the art. The optimal coating for any particular formulation depends on the exact intended use, and coatings may be tailored to release the active ingredient in a particular region of the intestines, or at a particular time following ingestion. Such a formulation may if desired contain one or more intermediate layers between the active ingredient and the outer enteric coating. In this case, it is possible for a composition of the invention to release a portion of its contents at one particular region of the intestine, and a further portion of its contents in a second region of the intestine, such as the colon. Preferably, the composition of the present invention is in a solid or semi-solid form which comprises an enteric coating adapted to release the protein or peptide in the colon. Useful enteric coatings are those which remain intact in the low pH environment of the stomach, but readily dissolve when the optimum pH for dissolution is reached. This can vary between pH 3 to 7.5, preferably 5 to 7, depending on the chemical composition of the coating. The thickness of the coating required will depend on the solubility of the coating and the intended site to be treated. Typically, the coating is 25 to 200 μm, especially 75 to 150 μm.

The composition of the invention is adapted for release of the active ingredient to the part of the lower gastrointestinal tract where the disease is prevalent. Typically, the enteric coating should dissolve in the pH of the jejunum (about pH 5.5), ileum (about pH 6) and/or colon (pH 6-7) to that the majority of the protein or peptide active ingredient is released at the desired site.

WO 2007/122374 (the contents of which are incorporated herein by reference) describes compositions for selective release within the colon, and these form one preferred embodiment of the invention. Accordingly, the invention further provides a composition comprising a particle with a core and a coating for the core, the core comprising a protein or peptide together with 5-ALA and optionally other stabilising agents such as a pH-modulating agent, and/or an enzyme inhibitor, the coating comprising a mixture of a mixture of a digestible polysaccharide and a film-forming material which has a solubility threshold at pH 6.0 or above, preferably pH 7 or above.

The digestible polysaccharide is susceptible to attack by intestinal bacteria. Preferably the digestible polysaccharide. The digestible polysaccharide is preferably selected from the group consisting of starch; amylose; amylopectin; chitosan; chondroitin sulfate; cyclodextrin; dextran; pullulan; carrageenan; scleroglucan; chitin; curdulan and levan.

For example, the polysaccharide may be starch, amylose or amylopectin.

The film-forming material is an enteric material which has a pH solubility threshold which is the pH below which it is insoluble and at or above which it is soluble. The pH of the surrounding medium triggers dissolution of the second material. The normal pH of gastric juice is usually in the range of 1 to 3, while the pH of intestinal juice gradually increases from about 5.5 in the duodenum to about 7 to 8 in the colon. Thus, the second material, when used in a composition of the present invention, has a pH solubility threshold of 6.0 or greater, especially 7 or greater.

The film-forming material is typically elected from an acrylate polymer, a cellulose polymer or a polyvinyl-based polymer. Examples of suitable cellulose polymers include cellulose acetate phthalate (“CAP”); cellulose acetate trimellitate (“CAT”); and hydropropylmethylcellulose acetate succinate. Examples of suitable polyvinyl-based polymers include polyvinyl acetate phthalate (“PVAP”). The film-forming material is preferably a co-polymer of a (meth)acrylic acid and a (meth)acrylic acid C₁₋₄ alkyl ester, for instance, a copolymer of methacrylic acid and methacrylic acid methyl ester. Such polymers include those available under the Trade Marks Eudragit L, Eudragit S and Eudragit FS. The use of Eudragit S as the film-forming material is particularly preferred.

In such compositions, multi-unit dosage forms comprising particles having a diameter of less than 3 mm are preferred. The “core” is usually a single solid body. The core may consist of the protein or peptide together with the 5-ALA and optionally a pH-modulating agent, and/or an enzyme inhibitor acting as a stabilising agent. More usually, however, the core will comprise a mixture of the protein or peptide and said 5-ALA, optionally with a pH-modulating agent, and/or an enzyme inhibitor, and further optionally one or more additional excipient. The core may for example include a filler or diluent material, e.g. lactose or cellulose material such as microcrystalline cellulose; a binder, e.g. polyvinylpyrrolidone (PVP); a disintegrant, e.g. croscarmellose sodium; and/or a lubricant, e.g. magnesium stearate. The core may be a compressed granulate comprising at least some of these materials.

Release from such compositions is delayed until the lower gastro-intestinal tract, in particular the ileum and/or the colon. Such compositions have application in a multi-phasic release composition comprising at least two pluralities of particles, e.g. coated pellets, in the same dosage form, e.g. a capsule, in which the particles of one plurality are differentiated from the particles of the or each other plurality by the coating. The coatings may differ from one plurality to the next in terms of coating thickness or composition, e.g. the ratio and/or identity of components. Multi-phasic release formulations would be particularly suitable for suffers of Crohn's disease affecting different regions along the intestine, including the ileum and/or the colon.

### Medical Applications

The present invention provides a pharmaceutical composition according to the invention for use in therapy. It also provides a method of treating or preventing a disease or condition in a subject, especially a human subject, which comprises administering to the subject via the lower gastro-intestinal tract, especially the ileum and/or the colon a pharmaceutical composition which comprises as active ingredient a protein or a peptide; 5-aminolevulinic acid (5-ALA) or a pharmaceutically acceptable salt thereof; and optionally a pH-modulating component and/or an enzyme inhibitor. Preferably, the compositions are adapted for administration via the oral or rectal route. Although the invention finds utility in the treatment of diseases of the lower gastro-intestinal tract, especially the ileum and/or the colon, it also has application as a portal for entry of a protein or peptide into the systemic circulation by absorption from the lower gastro-intestinal tract, especially the ileum and/or the colon, and hence finds utility in the treatment of a wide range of diseases and conditions.

The following non-limiting Examples illustrate the invention.

### Materials and Methods

### Human Colon Model

A human colonic model based on a mixed faecal inoculum was used to mimic the luminal environment of the human large intestine. An anaerobic workstation (Electrotek 500TG™ workstation, Electrotek, West Yorkshire, UK) maintained at 37° C. and 70% relative air humidity was used to set up the model. The faecal material was transferred in the anaerobic workstation and diluted with freshly prepared basal medium to obtain 25% w/w colon fluid by homogenization. The basal media provides nutrients and growth factors to the microbiota allowing viability for up to 24 hours. The homogenized bacterial media was sieved through an open mesh fabric (SefarNitex™, pore size 350 μm) to remove any nonhomogeneous fibrous material. The pH was maintained at approximately 7.0 to mimic the colonic luminal pH of the human.

### Rat Caecum Slurry

The rat caecum slurry was based on combination of cecum and proximal colon fluids collected from healthy wistar rats weighing 250 g. The fluids were collected immediately after sacrificing the rats and transferred to an anaerobic workstation (Electrotek 500TG™ workstation, Electrotek, West Yorkshire, UK) maintained at 37° C. and 70% relative air humidity. The fluids were diluted with freshly prepared basal medium to obtain a 50% w/w caecum fluid by homogenization. The homogenized mixture was sieved through an open mesh fabric. The pH of the fluid was measured as 5.5.

### Antibody Incubation Studies

Antibody stock solution (infliximab) was prepared in PBS at a 2 mg/ml concentration. 5-ALA was prepared at concentrations 10, 20 or 40 mg/ml. Methyl-ALA, hexyl-ALA, salcaprozate sodium, (SNAC) and sodium chenodeoxycholate were prepared at 20 mg/ml concentrations. The base L-arginine was prepared at 11, 22 or 44 mg/ml, and the stabiliser aprotinin was prepared at a 2 mg/ml concentration. Stock solutions containing certain infliximab combinations (Examples 1-5) were added to either 25% w/w human colon fluid (human colon model) or 50% w/w rat caecum slurry at a 1:1 dilution to obtain incubation concentrations of 1 mg/ml infliximab, 5, 10 or 20 mg/ml 5-ALA, 5.5, 11 or 22 mg/ml L-arginine, 10 mg/ml methyl-ALA, 10 mg/ml Hexyl-ALA, 10 mg/ml salcaprozate sodium, 10 mg/ml sodium chenodeoxycholate, and 12.5% w/w human colon fluid or 25% w/w rat caecum slurry.

Samples were withdrawn at appropriate time points and added to a protease inhibitor cocktail (Sigma-Aldrich, P2714) in a ratio of 1:3. The samples were centrifuged at 9.6 g for 10 mins and the supernatant was analysed by size exclusion-HPLC (SE-HPLC).

### Peptide Incubation Studies

Peptide stock solution (oxytocin) were prepared in PBS at 2 mg/ml concentrations. Concentrations of 10 mg/ml and 40 mg/ml 5-ALA, 40 mg/ml methyl-ALA, 40 mg/ml hexyl-ALA and 2 mg/ml aprotinin were prepared. Stock solutions containing certain oxytocin combinations were added to 50% rat caecum slurry to obtain incubation concentrations (Examples 6 and 7) of 1 mg/ml peptide, 5 mg/ml or 20 mg/ml 5-ALA, 20 mg/ml methyl-ALA, 20 mg/ml hexyl-ALA, 1 mg/ml aprotinin, and 25% w/w rat caecum slurry. Samples were withdrawn at appropriate time points and added to 0.1N HCl solution in a ratio of 1:3. The samples were centrifuged at 9.6 g for 10 mins and the supernatant was analysed by reversed phase-HPLC (RP-HPLC).

### Ussing Chamber System

A NaviCyte vertical Ussing system (Harvard Apparatus, Cambridge, UK) was used to measure transport across epithelial membranes which are polar structures possessing an apical (mucosal) and basolateral (serosal) side. The chambers are made of solid acrylic and supports the tissue membrane in such a way that each side of the membrane is isolated and faces a different chamber representing the luminal (apical) and blood (basal) compartments. The working system consists of a unit to fit a maximum of six vertical chambers, a gas manifold for carbogen purging (95% O₂, 5% CO₂) and a heater block to maintain the temperature of the chambers at 37° C. during the experiments with the use of a circulating water bath. The chambers are two-piece assemblies held together by a high spring-tension retaining ring to ensure leak-free operation during the experiments.

The EVOM™ voltohmmeter (World Precision Instruments, Inc., Hertfordshire, UK) and Ag/AgCl electrodes (Harvard Apparatus, Cambridge, UK) were used to measure the trans-epithelial electrical resistance (TEER) of the tissue samples. TEER monitors the presence of functional tight junctions, which are responsible for the barrier function and which limit paracellular permeation of water and solutes. TEER value of 200 Ω/cm² was set as the lower limit to confirm the tissue viability and tight junction integrity.

For the tissue penetration studies, the freshly excised small intestine (jejunum) and proximal colon tissue of healthy wistar rats was collected and transferred to an ice-cold solution of Krebs-Bicarbonate Ringer solution (KBr) of pH 7.4. The tissue was cut open transversally and was washed with KBr solution to remove the luminal contents and was then mounted in the Ussing chambers. The mucosal surface of the colon tissue was facing the apical chamber, and the endothelial surface of the tissue was facing the basolateral chamber. The exposed tissue area on each side of the chamber was 0.29 cm² and the tissue mounting region was 4×8 mm. The volume of KBr in apical and basolateral chamber was 5 ml and the pH was maintained at 7.4. The tissue was allowed to incubate with KBr for 15 minutes before addition of the drug. The antibody and peptides; infliximab and oxytocin were tested in certain combinations (Examples 8-14), with and without-ALA, methyl-ALA, hexyl-ALA, L-arginine, sodium caprate, sodium chenodeoxycholate, SNAC, labrasol, Na₂EDTA and N-acetylcysteine (NAC). The drug concentrations tested during the penetration experiments were 0.2 mg/ml and 1 mg/ml for antibody and peptide studies, respectively. The penetration of antibody/peptide in the small intestine and colon tissue was tested for 2 hours and in a minimum of 2 rats. The tissue without drug was incubated in parallel for the same time which acted as the negative control. The chambers were purged with carbogen and kept at 37° C. by water jackets during incubation. The TEER was continuously monitored during the experiment to confirm the viability and integrity of the tissue. Tissues with TEER value below 200 Ω/cm² were not used for the experiments.

### SE-HPLC

Infliximab sample analysis (stability studies) was performed using size exclusion-high performance liquid chromatography (SE-HPLC) system (Agilent Technologies, 1260 Infinity II Series™) equipped with a pump (model G7111A), autosampler (model G7129A) and a diode-array UV detector (model G7114A). A 300×8.0-mm YMC-Pack Diol-200 S-5 μm, 20 nm (YMC Co Ltd, Kyoto, Japan) size exclusion (SE) chromatography column was used for sample separation using phosphate buffer saline (pH 7.3) prepared in sterile HPLC grade water as the mobile phase for elution, at a flow rate of 0.5 ml/min. The analysis was operated at room temperature and UV detection wavelength was set at 214 nm. Each sample was run for 40 minutes to allow complete elution of the sample proteins and reduce run-over. The retention time for infliximab was 15 minutes.

### RP-HPLC

Peptide sample analysis (stability and permeability studies) was performed using a reversed phase liquid chromatography (RP-HPLC) system (Agilent Technologies, 1260 Infinity II Series™) equipped with a pump (model G7111A), autosampler (G7129A) and a diode-array UV detector (model G7114A). A 150×4.6-mm Luna 5 μm C18 100 Å (Phenomenex, Torrance, CA) column was used using a gradient system as below:

| | % Mobile Phase | % Mobile |
|---|---|---|
| Time | A: 0.1% TFA | Phase C: |
| (mins) | in Water | Acetonitrile |
| 0.00 | 83.0 | 17.0 |
| 4.00 | 83.0 | 17.0 |
| 20.00 | 70.0 | 30.0 |
| 20.01 | 83.0 | 17.0 |
| 23.00 | 83.0 | 17.0 |

Each sample's injection volume was 20 μl, the mobile phase flow rate was 1 ml/min and the peptide was detected at 214 nm. Each sample was run for 23 mins.

### ELISA

Infliximab sample analysis (permeability studies) was performed using an indirect ELISA platform assay. A 96-well plate was coated at 1 ug/ml TNF-α. 5% BSA in PBS-T was used to block the plate to avoid non-specific binding. The drug samples were incubated for 1 h at 37° C. An Fc-specific secondary antibody was added to bind specifically to infliximab. TMB substrate was added to the wells and allowed to incubate in the dark to promote a colour change depending on level of antibody binding, before addition of stop solution. Between each step, the plate was washed with 0.1% Tween 20 in PBS. The plate was read at a wavelength of 650 nm with the plate reader (Synergy LX Multimode Reader, Biotek).

### Example 1. Stability of the Monoclonal Antibody, Infliximab in the Presence of 5-ALA

Stability of the drug/antibody infliximab in human colon fluid was measured with and without 5-ALA at varying concentrations. Each experiment was conducted in duplicate/triplicate. The antibody remaining at different time points was measured by SE-HPLC. The results are shown in the table below.

| | | | Fold- | | Fold- |
|---|---|---|---|---|---|
| | Incubation | % Drug at | increase | % Drug at | increase |
| Formulation | Concentration | T = 2 h | at 2 h* | T = 4 h | at 4 h* |
| Infliximab | 1 mg/ml | 21.5 ± 0.0 | — | 7.8 ± 0.0 | — |
| (pH 6.5) | | | | | |
| Infliximab + | 1 mg/ml + | 59.8 ± 5.2 | 3.6-fold | 17.5 ± 2.8 | 2.7-fold |
| 5-ALA.HCl | 5 mg/ml | | | | |
| (pH 5.5) | | | | | |
| Infliximab + | 1 mg/ml + | 42.3 ± 0.5 | 2.6-fold | 13.8 ± 0.2 | 2.0-fold |
| 5-ALA.HCl | 10 mg/ml | | | | |
| (pH 4.9) | | | | | |
| Infliximab + | 1 mg/ml + | 16.5 ± 0.4 | — | 2.3 ± 0.3 | — |
| 5-ALA.HCl | 20 mg/ml | | | | |
| (pH 4.1) | | | | | |

*Compared to infliximab alone.

The results are shown in the table above and demonstrate that 5-ALA provides antibody stability. However, an increase in concentration of 5-ALA leads to a reduction in pH of the drug solution. 5-ALA does not stabilize the antibody in a dose dependent manner.

### Example 2. Stability of the Monoclonal Antibody, Infliximab in the Presence of 5-ALA and the Base, L-Arginine

Stability of the drug infliximab in human colon fluid was measured with and without 5-ALA and L-arginine at varying concentrations. Each experiment was conducted in duplicate/triplicate. The antibody remaining at different time points was measured by SE-HPLC.

| | | | Fold- | | Fold- |
|---|---|---|---|---|---|
| | Incubation | % Drug at | increase | % Drug at | increase |
| Formulation | Concentration | T = 2 h | at 2 h* | T = 4 h | at 4 h* |
| Infliximab (pH 6.5) | 1 mg/ml | 16.5 ± 0.4 | — | 6.5 ± 0.3 | — |
| Infliximab + 5-ALA | 1 mg/ml + 5 mg/ml + | 46.7 ± 1.5 | 2.8-fold | 25.5 ± 1.3 | 3.9-fold |
| phosphate + L-arginine (pH 6.4) | 5.5 mg/ml | | | | |
| Infliximab + 5-ALA | 1 mg/ml + 10 mg/ml + | 83.2 ± 2.8 | 5.0-fold | 37.0 ± 0.8 | 5.7-fold |
| phosphate + L-arginine (6.5) | 11 mg/ml | | | | |
| Infliximab + 5-ALA | 1 mg/ml + 20 mg/ml + | 85.7 ± 2.9 | 5.2-fold | 43.2 ± 2.0 | 6.5-fold |
| phosphate + L-arginine (pH 6.5) | 22 mg/ml | | | | |
| Infliximab + 5-ALA | 1 mg/ml + 20 mg/ml | 76.4 ± 6.5 | 4.6-fold | 33.1 ± 3.8 | 5.1-fold |
| phosphate + NaOH (pH 6.4) | | | | | |

*Compared to infliximab alone.

The results are shown in the table above and demonstrate that 5-ALA with increasing concentrations in the presence of a pH modulating component, L-arginine, helps to act as a stabiliser and increase the amount of the antibody present in the colon fluid.

### Example 3. Stability of the Monoclonal Antibody, Infliximab in the Presence of 5-ALA and Aprotinin

Stability of the drug infliximab in human colon fluid was measured with and without 5-ALA and aprotinin at varying pH. Each experiment was conducted in duplicate/triplicate. The antibody remaining at different time points was measured by SE-HPLC.

| | | | Fold- | | Fold- |
|---|---|---|---|---|---|
| | Incubation | % Drug at | increase | % Drug at | increase |
| Formulation | Concentration | T = 2 h | at 2 h* | T = 4 h | at 4 h* |
| Infliximab + 5-ALA.HCl | 1 mg/ml + 20 mg/ml | 79.0 ± 0.1 | 4.8-fold | 64.0 ± 0.6 | 9.8-fold |
| (pH 6.5) | | | | | |
| Infliximab + 5-ALA.HCl + | 1 mg/ml + 20 mg/ml + | 104.0 ± 0.8 | 6.3-fold | 95.0 ± 9.5 | 14.6-fold |
| Aprotinin (pH 6.5) | 1 mg/ml | | | | |
| Infliximab + 5-ALA.HCl | 1 mg/ml + 20 mg/ml | 54.0 ± 4.1 | 3.3-fold | 32.0 ± 1.7 | 4.9-fold |
| (pH 7.8) | | | | | |
| Infliximab + 5-ALA.HCl + | 1 mg/ml + 20 mg/ml + | 98.0 ± 2.8 | 5.9-fold | 103.0 ± 4.4 | 15.8-fold |
| Aprotinin (pH 7.8) | 1 mg/ml | | | | |

*Compared to infliximab alone.

The results are shown in the table above and demonstrate that 5-ALA helps to act as a stabiliser and increase the amount of the antibody present in the colon fluid, and to a greater extent with addition of an enzyme inhibitor, aprotinin. pH 6.5 is ideal for antibody stabilisation, as there is a reduction in stability with increasing pH to 7.8.

### Example 4. Stability of the Monoclonal Antibody, Infliximab in the Presence of 5-ALA, 5-ALA Lipophilic Derivatives and Aprotinin

Stability of the drug infliximab in human colon fluid was measured with and without 5-ALA, methyl-ALA and hexyl-ALA and aprotinin. Each experiment was conducted in duplicate/triplicate. The antibody remaining at different time points was measured by SE-HPLC.

| | | | Fold- | | Fold- |
|---|---|---|---|---|---|
| | Incubation | % Drug at | increase | % Drug at | increase |
| Formulation | Concentration | T = 2 h | at 2 h* | T = 4 h | at 4 h* |
| Infliximab (pH 6.5) | 1 mg/ml | 37.6 ± 3.5 | — | 16.1 ± 0.6 | — |
| Infliximab + 5-ALA.HCl | 1 mg/ml + 10 mg/ml | 82.5 ± 5.0 | 2.2-fold | 49.9 ± 7.4 | 3.1-fold |
| (pH 6.5) | | | | | |
| Infliximab + 5-ALA.HCl + | 1 mg/ml + 10 mg/ml + | 91.3 ± 15.5 | 2.4-fold | 83.1 ± 7.2 | 5.2-fold |
| Aprotinin (pH 6.5) | 1 mg/ml | | | | |
| Infliximab + Methyl-ALA | 1 mg/ml + 10 mg/ml | 92.0 ± 1.0 | 2.4-fold | 68.0 ± 8.0 | 4.2-fold |
| (pH 6.5) | | | | | |
| Infliximab + Methyl-ALA + | 1 mg/ml + 10 mg/ml + | 106.0 ± 2.0 | 2.8-fold | 86.0 ± 3.0 | 5.3-fold |
| Aprotinin (pH 6.5) | 1 mg/ml | | | | |
| Infliximab + Hexyl-ALA | 1 mg/ml + 10 mg/ml | 90.0 ± 17.1 | 2.4-fold | 55.2 ± 13.2 | 3.4-fold |
| (pH 6.5) | | | | | |
| Infliximab + Hexyl-ALA + | 1 mg/ml + 10 mg/ml + | 126.0 ± 0.5 | 3.4-fold | 125.0 ± 6.7 | 7.8-fold |
| Aprotinin (pH 6.5) | 1 mg/ml | | | | |

*Compared to infliximab alone.

The results are shown in the table above and demonstrate that 5-ALA helps to act as a stabiliser and increase the amount of the antibody present in the colon fluid. 5-ALA lipophilic derivatives; methyl-ALA and hexyl-ALA were tested and shown to offer greater antibody stabilisation, and to a greater extent with the addition of aprotinin.

### Example 5. Stability of the Monoclonal Antibody, Infliximab in the Presence of Permeation Enhancers, a 5-ALA Lipophilic Derivative and Aprotinin

Stability of the drug infliximab in rat caecum slurry was measured with and without SNAC, sodium chenodeoxycholate, hexyl-ALA and aprotinin. Each experiment was conducted in duplicate/triplicate. The antibody remaining at different time points was measured by SE-HPLC.

| | | | Fold- | | Fold- |
|---|---|---|---|---|---|
| | Incubation | % Drug at | increase | % Drug at | increase |
| Formulation | Concentration | T = 2 h | at 2 h* | T = 4 h | at 4 h* |
| Infliximab (pH 6.4) | 1 mg/ml | 47.7 ± 0.0 | — | 8.4 ± 0.0 | — |
| Infliximab + | 1 mg/ml + 1 mg/ml | 102.0 ± 0.0 | 2.1-fold | 62.5 ± 0.0 | 7.4-fold |
| Aprotinin (pH 6.4) | | | | | |
| Infliximab + SNAC (pH 6.5) | 1 mg/ml + 10 mg/ml | 34.0 ± 0.0 | 0.7-fold | 4.0 ± 0.0 | 0.5-fold |
| Infliximab + SNAC + | 1 mg/ml + 10 mg/ml + | 107.8 ± 0.1 | 2.3-fold | 71.3 ± 0.1 | 8.5-fold |
| Aprotinin (pH 6.5) | 1 mg/ml | | | | |
| Infliximab + Sodium | 1 mg/ml + 10 mg/ml | 13.9 ± 0.0 | 0.3-fold | 0.0 | — |
| chenodeoxycholate (pH 6.8) | | | | | |
| Infliximab + Sodium | 1 mg/ml + 10 mg/ml + | 84.5 ± 0.0 | 1.8-fold | 62.3 ± 0.0 | 7.3-fold |
| chenodeoxycholate + | 1 mg/ml | | | | |
| Aprotinin (pH 6.8) | | | | | |
| Infliximab + | 1 mg/ml + 10 mg/ml | 92.6 ± 0 | 1.9-fold | 54.7 ± 0.0 | 6.5-fold |
| Hexyl-ALA (pH 5.5) | | | | | |
| Infliximab + Hexyl-ALA + | 1 mg/ml + 10 mg/ml + | 105.2 ± 0.1 | 2.2-fold | 101.3 ± 0.0 | 12.0-fold |
| Aprotinin (pH 5.6) | 1 mg/ml | | | | |

*Compared to infliximab alone.

The results are shown in the table above and demonstrate aprotinin alone provides an antibody stabilisation effect, the permeation enhancers, SNAC and sodium chenodeoxycholate alone provide no stabilisation, and hexyl-ALA both alone and with aprotinin provide good antibody stability.

### Example 6. Stability of the Peptide Drug Oxytocin in the Presence of 5-ALA and Aprotinin

Stability of the drug oxytocin in rat caecum slurry was measured with and without 5-ALA and aprotinin, an enzyme inhibitor. Each experiment was conducted in duplicate/triplicate. The oxytocin remaining at different time points was measured by RP-HPLC.

| | | % | Fold- |
|---|---|---|---|
| | Incubation | Drug at | increase |
| Formulation | Concentration | T = 2 h* | at 2 h* |
| Oxytocin (pH 6.5) | 1 mg/ml | 10.9 ± 3.4 | — |
| Oxytocin + aprotinin | 1 mg/ml + 1 mg/ml | 14.2 ± 1.4 | 1.3-fold |
| (pH 6.5) | | | |
| Oxytocin + 5-ALA.HCl | 1 mg/ml + 20 mg/ml | 72.1 ± 2.5 | 6.6-fold |
| (pH 2.9) | | | |
| Oxytocin + 5-ALA.HCl + | 1 mg/ml + 20 mg/ml + | 73.1 ± 0.6 | 6.7-fold |
| aprotinin (pH 2.9) | 1 mg/ml | | |

*Compared to oxytocin alone.

The results are shown in the table above, and demonstrate that the 5-ALA acts as a stabiliser and increases the amount of peptide present in the caecum slurry, with slight improvement in oxytocin stability with addition of aprotinin.

### Example 7. Stability of the Peptide Oxytocin in the Presence of 5-ALA, 5-ALA Lipophilic Derivatives and Aprotinin

Stability of the drug oxytocin in rat caecum slurry was measured with and without methyl-ALA, hexyl-ALA and aprotinin, an enzyme inhibitor. Each experiment was conducted in duplicate/triplicate. The oxytocin remaining at different time points was measured by RP-HPLC.

| | | % | Fold- |
|---|---|---|---|
| | Incubation | Drug at | increase |
| Formulation | Concentration | T = 2 h* | at 2 h* |
| Oxytocin (pH 6.5) | 1 mg/ml | 5.0 ± 2.0 | — |
| Oxytocin + Methyl-ALA | 1 mg/ml + 20 mg/ml | 12.0 ± 0.0 | 2.4-fold |
| (pH 6.3) | | | |
| Oxytocin + Methyl-ALA + | 1 mg/ml + 20 mg/ml + | 15.0 ± 1.0 | 3.0-fold |
| Aprotinin (pH 6.3) | 1 mg/ml | | |
| Oxytocin + Hexyl-ALA | 1 mg/ml + 20 mg/ml | 26.0 ± 1.0 | 5.2-fold |
| (pH 5.7) | | | |
| Oxytocin + Hexyl-ALA + | 1 mg/ml + 20 mg/ml + | 30.0 ± 1.0 | 6.0-fold |
| Aprotinin (pH 5.7) | 1 mg/ml | | |

*Compared to oxytocin alone.

The results are shown in the table above, and demonstrate that methyl-ALA and hexyl-ALA provide some peptide stabilisation (though not as much as 5-ALA—see Example 6), with a slight increase in stabilisation with addition of aprotinin in caecum slurry. As 5-ALA naturally reduces the pH of oxytocin solution, which helps in peptide stabilization, its lipophilic derivatives do not provide significant reductions in pH, which therefore result in lower stability profiles.

### Example 8. Permeation of the Antibody, Infliximab Through GI Tissue in the Presence of 5-ALA and the Base L-Arginine

Permeability of the drug infliximab across rat small intestine (jejunum) and colon tissue was measured with and without 5-ALA and the base, L-arginine. The antibody in the tissue at 2 h was measured by ELISA.

| | | % Drug in | |
|---|---|---|---|
| | | small | % Drug in |
| | Incubation | intestine | colon tissue |
| Formulation | Concentration | tissue at 2 h | at 2 h |
| Infliximab | 0.2 mg/ml | — | 4.7 ± 0.033 |
| Infliximab + | 0.2 mg/ml + | — | 2.5 ± 0.019 |
| 5-ALA.HCl | 4 mg/ml | | |
| Infliximab + | 0.2 mg/ml + | — | 1.7 ± 0.014 |
| 5-ALA.HCl + | 4 mg/ml + | | |
| L-arginine | 4.4 mg/ml | | |
| Infliximab | 0.2 mg/ml | 2.0 ± 0.005 | — |
| Infliximab + | 0.2 mg/ml + | 1.2 ± 0.010 | — |
| 5-ALA.HCl | 4 mg/ml | | |
| Infliximab + | 0.2 mg/ml + | 1.6 ± 0.004 | — |
| 5-ALA.HCl + | 4 mg/ml + | | |
| L-arginine | 4.4 mg/ml | | |

The results are shown in the table above, and demonstrate that 5-ALA with or without the base, L-arginine did not lead to permeation or improved tissue uptake of infliximab in neither small intestine nor colon tissue.

### Example 9. Permeation of the Antibody, Infliximab Through GI Tissue in the Presence Permeation Enhancers

Permeability of the drug infliximab across rat small intestine (jejunum) was measured with and without sodium caprate, SNAC, sodium chenodeoxycholate, labrasol and Na₂EDTA. The antibody in the tissue at 2 h was measured by ELISA.

| | | % Drug in |
|---|---|---|
| | Incubation | small intestine |
| Formulation | Concentration | tissue at 2 h |
| Infliximab | 0.2 mg/ml | 1.6 ± 0.011 |
| Infliximab + Sodium caprate | 0.2 mg/ml + 4 mg/ml | 2.4 ± 0.005 |
| Infliximab + SNAC | 0.2 mg/ml + 6 mg/ml | 2.1 ± 0.006 |
| Infliximab + Sodium | 0.2 mg/ml + 9 mg/ml | 1.2 ± 0.002 |
| chenodeoxycholate | | |
| Infliximab + Labrasol | 0.2 mg/ml + 8 mg/ml | 2.8 ± 0.020 |
| Infliximab + Na₂EDTA | 0.2 mg/ml + 7 mg/ml | 2.1 ± 0.004 |

The results are shown in the table above, and demonstrate that there was some increase in infliximab tissue levels with all permeation enhancers, except sodium chenodeoxycholate.

### Example 10. Permeation of the Antibody, Infliximab Through GI Tissue in the Presence of a 5-ALA Lipophilic Derivative

Permeability of infliximab across rat small intestine (jejunum) was measured with and without hexyl-ALA. The antibody in the tissue at 2 h was measured by ELISA.

| | | % Drug in |
|---|---|---|
| | Incubation | small intestine |
| Formulation | Concentration | tissue at 2 h |
| Infliximab | 0.2 mg/ml | 1.7 ± 0.006 |
| Infliximab + Hexyl-ALA | 0.2 mg/ml + 4 mg/ml | 2.4 ± 0.004 |

The results are shown in the table above, and demonstrate that there was some increase in infliximab tissue levels with hexyl-ALA.

### Example 11. Permeation of the Antibody, Infliximab Through GI Tissue in the Presence of a 5-ALA Lipophilic Derivative, Permeation Enhancers and N-Acetylcysteine

Permeability of the drug infliximab across rat colon tissue was measured with and without sodium caprate, SNAC, sodium chenodeoxycholate and hexyl-ALA. N-Acetylcysteine (NAC) was applied to the tissue prior to addition of antibody to stimulate tissue damage. The antibody in the tissue at 2 h was measured by ELISA.

| | | % Drug in |
|---|---|---|
| | Incubation | colon tissue |
| Formulation | Concentration | at 2 h |
| Infliximab + NAC | 0.2 mg/ml | 2.1 ± 0.008 |
| Infliximab + Sodium | 0.2 mg/ml + 4 mg/ml | 2.8 ± 0.016 |
| caprate + NAC | | |
| Infliximab + SNAC + NAC | 0.2 mg/ml + 6 mg/ml | 3.6 ± 0.020 |
| Infliximab + Sodium | 0.2 mg/ml + 9 mg/ml | 2.8 ± 0.017 |
| chenodeoxycholate + NAC | | |
| Infliximab + | 0.2 mg/ml + 4 mg/ml | 2.0 ± 0.015 |
| Hexyl-ALA + NAC | | |

The results are shown in the table above, and demonstrate that using NAC to mimic diseased tissue conditions may have led to increased tissue levels of antibody through sodium caprate, SNAC, sodium chenodeoxycholate and hexyl-ALA.

### Example 12. Permeation of Oxytocin Through GI Tissue in the Presence of 5-ALA

Permeability of the drug oxytocin across rat small intestine (jejunum) and colon tissue was measured with and without 5-ALA. The peptide in the tissue at 2 h and was measured by RP-HPLC.

| | | % Drug in | |
|---|---|---|---|
| | | small | % Drug in |
| | Incubation | intestine | colon tissue |
| Formulation | Concentration | tissue at 2 h | at 2 h |
| Oxytocin | 1 mg/ml | — | 0.0 ± 0.0 |
| Oxytocin + | 1 mg/ml + | — | 0.6 ± 0.002 |
| 5-ALA.HCl | 20 mg/ml | | |
| Oxytocin | 1 mg/ml | 0.0 ± 0.0 | — |
| Oxytocin + | 1 mg/ml + | 0.5 ± 0.001 | — |
| 5-ALA.HCl | 20 mg/ml | | |

The results are shown in the table above, and demonstrate that 5-ALA acts as a permeability enhancer and increases the amount of oxytocin which is present in both small intestinal tissue and colon tissue.

### Example 13. Permeation of Oxytocin Through GI Tissue in the Presence of 5-ALA and 5-ALA Lipophilic Derivatives

Permeability of oxytocin across rat small intestine (jejunum) was measured with and without 5-ALA, methyl-ALA and hexyl-ALA. The peptide in the tissue at 2 h and was measured by RP-HPLC.

| | | % Drug in |
|---|---|---|
| | Incubation | small intestine |
| Formulation | Concentration | tissue at 2 h |
| Oxytocin | 1 mg/ml | 0.1 ± 0.0 |
| Oxytocin + 5-ALA.HCl | 1 mg/ml + 20 mg/ml | 0.5 ± 0.001 |
| Oxytocin + Methyl-ALA | 1 mg/ml + 20 mg/ml | 0.4 ± 0.002 |
| Oxytocin + Hexyl-ALA | 1 mg/ml + 20 mg/ml | 0.3 ± 0.001 |

The results are shown in the table above, and demonstrate that 5-ALA acts as a permeability enhancer and increases the amount of oxytocin which is present in the small intestinal tissue. The lipophilic derivatives, methyl-ALA and hexyl-ALA also show enhanced permeation of oxytocin, but to a lesser extent compared to 5-ALA.

### Example 14. Permeation of Oxytocin Through GI Tissue in the Presence of Permeation Enhancers and a 5-ALA Lipophilic Derivative

Permeability of oxytocin across rat small intestine (jejunum) was measured with and without, sodium caprate, SNAC, labrasol, methyl-ALA and hexyl-ALA. The peptide in the tissue at 2 h and was measured by RP-HPLC.

| | | % Drug in |
|---|---|---|
| | Incubation | small intestine |
| Formulation | Concentration | tissue at 2 h |
| Oxytocin | 1 mg/ml | 0.1 ± 0.001 |
| Oxytocin + Sodium caprate | 1 mg/ml + 20 mg/ml | 0.3 ± 0.001 |
| Oxytocin + SNAC | 1 mg/ml + 20 mg/ml | 0.5 ± 0.001 |
| Oxytocin + Labrasol | 1 mg/ml + 20 mg/ml | 0.2 ± 0.0 |
| Oxytocin + Methyl-ALA | 1 mg/ml + 20 mg/ml | 0.2 ± 0.001 |
| Oxytocin + Hexyl-ALA | 1 mg/ml + 20 mg/ml | 0.2 ± 0.0 |

The results are shown in the table above, and demonstrate that the Permeation Enhancers and 5-ALA lipophilic derivatives act as permeability enhancers and increases the amount of oxytocin which is present in the small intestinal tissue. Sodium caprate and SNAC showed the greatest increase in oxytocin tissue levels.

1. A pharmaceutical composition comprising (a) as active ingredient a protein or a peptide and (b) 5-aminolevulinic acid (5-ALA) or a pharmaceutically acceptable salt thereof, and optionally (c) a pH-modulating component and/or (d) an enzyme inhibitor.

2. A pharmaceutical composition as claimed in claim 1, wherein the active ingredient is a protein.

3. A pharmaceutical composition as claimed in claim 2, wherein the active ingredient is an antibody.

4. A pharmaceutical composition as claimed in claim 1, wherein the active ingredient is a peptide.

5. A pharmaceutical composition as claimed in any preceding claim, wherein the enzyme inhibitor is aprotinin.

6. A pharmaceutical composition as claimed in any preceding claim in a liquid, solid or semi-solid form suitable for oral or rectal administration.

7. A pharmaceutical composition as claimed in any preceding claim in a solid form suitable for oral administration, said composition having an enteric coating.

8. A pharmaceutical composition as claimed in any preceding claim adapted for selective release of the peptide or protein in the lower gastro-intestinal tract.

9. A pharmaceutical composition as claimed in any one of the preceding claims comprising a solid dosage form with a core and a coating for the core, the core comprising the active ingredient, the 5-aminolevulinic acid and optionally the base or enzyme inhibitor, and the coating comprising a mixture of a digestible polysaccharide and a film-forming material which has a solubility threshold at pH 6.0 or above.

10. A pharmaceutical composition as claimed in claim 9, wherein the digestible polysaccharide is selected from the group consisting of starch; amylose; amylopectin; chitosan; chondroitin sulfate; cyclodextrin; dextran; pullulan; carrageenan; scleroglucan; chitin; curdulan and levan;

11. A pharmaceutical composition as claimed in claims 9 and 10, in which the film-forming material is an acrylate polymer, a cellulose polymer or a polyvinyl-based polymer.

12. A pharmaceutical composition as claimed in claim 11, in which the film-forming material is selected from cellulose acetate phthalate; cellulose acetate trimellitate; hydropropylmethylcellulose acetate succinate; and polyvinyl acetate phthalate.

13. An orally administrable pharmaceutical composition comprising (a) as active ingredient a protein or a peptide and (b) 5-aminolevulinic acid (5-ALA) or a pharmaceutically acceptable salt thereof.

14. A solid dosage form for oral administration comprising a core comprising (a) as active ingredient a protein or a peptide and (b) 5-aminolevulinic acid (5-ALA) or a pharmaceutically acceptable salt thereof, and (c) a pH-modulating component and/or (d) an enzyme inhibitor.

15. A rectally administrable pharmaceutical composition comprising (a) as active ingredient a protein or a peptide and (b) 5-aminolevulinic acid (5-ALA) or a pharmaceutically acceptable salt thereof, and (c) a pH-modulating component and/or (d) an enzyme inhibitor.

16. An enema formulation comprising (a) as active ingredient a protein or a peptide and (b) 5-aminolevulinic acid (5-ALA) or a pharmaceutically acceptable salt thereof, and and (c) a pH-modulating component and/or (d) an enzyme inhibitor.

17. A method of treating or preventing a disease or condition in a subject which comprises administering to the subject via the ileum and/or the colon a pharmaceutical composition which comprises (a) as active ingredient a protein or a peptide and (b) 5-aminolevulinic acid (5-ALA) or a pharmaceutically acceptable salt thereof, and and (c) a pH-modulating component and/or (d) an enzyme inhibitor.

18. A method as claimed in claim 16 or claim 17, wherein the disease or condition is inflammatory bowel disease; irritable bowel syndrome; constipation; diarrhoea; infection; autoimmune disease or cancer.

19. A composition as claimed in any one of claims 1 to 13 for use in therapy.

20. A method of stabilising a protein or a peptide in the presence of intestinal fluid, which comprises contacting the protein or peptide with 5-aminolevulinic acid (5-ALA) or a pharmaceutically acceptable salt thereof.

21. The use of 5-aminolevulinic acid (5-ALA) or a pharmaceutically acceptable salt thereof for the stabilisation of an active ingredient comprising a protein or peptide, which has been administered as a pharmaceutical composition and delivered to the lower gastro-intestinal tract.

22. The use of 5-aminolevulinic acid (5-ALA) or a pharmaceutically acceptable salt thereof to increase the permeability of the colon resulting in increased uptake of a protein or peptide which has been administered as a pharmaceutical composition and delivered to the lower gastro-intestinal tract.
